(19) United States
(12) Patent Application Publication
(10) Pub. No.: US 2006/0178311 A1
(43) Pub. Date: Aug. 10, 2006

(54) Control of lactation

(75) Inventors: Colin James Wilde, Ayr (GB); Malcolm Peaker, Ayr (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Hannah Research Institute, Ayr (GB)

(21) Appl. No.: 10/561,059

(22) PCT Filed: Jun. 16, 2004
(86) PCT No.: PCT/GB04/02558

§ 371 Date: Feb. 3, 2006

(30) Foreign Application Priority Data
Jun. 16, 2003 (GB) 0313892.2

(51) Int. Cl. A61K 38/08; A61K 38/10; C07K 7/08

(52) U.S. Cl. 514/15; 530/328

(57) ABSTRACT

The present invention provides three novel peptides identified from cow's milk and further to compositions and methods containing the same for use in the modulation of the milk secretion rate of a lactating cell. Specifically, at least one of the peptides provided by the invention can be used to control milk secretion in lactating animals, including man. The present invention further extends to antibodies directed to the peptides of the invention, said antibodies also having utility in the modulation of milk secretion.

### FIELD OF THE INVENTION

The present invention relates to the identification of three peptides which have a regulatory role in the control of milk secretion. The present invention further provides for the use of the identified peptides and antibodies thereto for the control of milk secretion in lactating animals, including humans.

### BACKGROUND OF THE INVENTION

Constituents of milk are known to control the rate of milk secretion according to the frequency and completeness with which those constituents are removed through the demand of the offspring or the farmer's husbandry. This biochemical feedback within the breast or udder acts to modulate the lactation promoting effects of galactopoietic hormones, and its regulatory characteristics, however not all the active factors in milk have been described by studies on lactating ruminants.

It has been previously shown that one factor active in goat and cow's milk is a 7.0-7.6 kDa protein present in a whey protein extract of milk from these animals. This factor has been shown to decrease lactose and casein synthesis in cultured explanted pieces of rabbit mammary tissue, and to decrease temporarily the rate of milk secretion when injected into a single mammary gland of the same species via the teat canal.

These studies did not demonstrate a relationship between the concentration of the 7.0-7.6 kDa protein in cow's milk and the animal's rate of milk secretion, and therefore no pivotal role for this protein in the feedback control of milk secretion by milk removal was demonstrated. It has remained a challenge to determine whether there are other inhibitory factors present in cow's milk which act to regulate supply of milk with demand through a process of feedback inhibition.

### SUMMARY OF THE INVENTION

According to a first aspect of the invention there is provided a peptide including the amino acid sequence RPKHPIKHQG (SEQ ID NO:1), AVAVSQEAN (SEQ ID NO:2) or SEGVALDPAR (SEQ ID NO:3) or an analogue thereof.

Preferably the peptides are isolated peptides.

As used herein, an “isolated” peptide is a peptide which is synthetic (e.g., recombinant), or which is altered, removed or purified from the natural state through human intervention.

Preferably the peptide is combined with at least one other of the two peptides including the amino acid sequence shown in SEQ ID NO:1, SEQ ID NO:2 or SEQ ID NO:3, this combination reducing milk secretion in animals, including humans.

Preferably the amino acid sequence shown in SEQ ID NO:1, SEQ ID NO: 2 and SEQ ID NO:3 is the N-terminal sequence of the peptide.

Preferably the peptide can be co-purified with each of the other peptides including the amino acid sequence shown in SEQ ID NO:1, SEQ ID NO:2 or SEQ ID NO:3 from a 6-30 kDa fraction of whey protein of cow's milk.

In particular, the peptides can be purified from cow's milk by a series of chromatographic separation techniques.

Specifically, when a 6-30 kDa fraction of the whey proteins of cow's milk is resolved by gel filtration on a cross-linked copolymer of allyl dextran and N,N methylenebisacrylamide having an average particle size of 47 μm, such as Sephacryl S-100 (Pharmacia), the fourth-eluting component resolved by this method, “peak S4”, comprises the inhibitory peptides.

More specifically, the peptides are co-purified when a nominally 6-30 kDa fraction of the whey proteins of cow's milk is resolved by gel filtration on a cross-linked copolymer of allyl dextran and N,N methylenebisacrylamide having an average particle size of 47 μm, such as Sephacryl S-100 (Pharmacia). The fourth-eluting component resolved by this method, “peak S4”, comprises the peptides.

When peak S4 is resolved further by peptide gel-filtration chromatography on a gel of dextran covalently bonded to highly cross-linked agarose beads with a mean diameter of 13-15 μm, such as Superdex Peptide HR (Pharmacia), the leading edge of the major eluted component eluting at 8-11.5 ml, designated P8-11A, contains the inhibitory peptides. Further, when fraction P8-11A is resolved by reversed phase chromatography on a reversed phase column (Genesis 25 cm, C18 4micron; Jones Chromatography), the fractions eluted after 34-36 minutes at a concentration of 36-39% acetonitrile, in a linear gradient of same in 0.1% trifluoroacetic acid, contains the peptides.

Preferably the amino acid sequence has at least about 30%, or 40%, or 50%, or 60%, or 70%, or 75%, or 80%, or 85%, or 90%, or 95% homology to the sequence shown in any one of SEQ ID NO:1, 2 or 3. Thus, a peptide fragment of any one of the peptides of the invention may include 1, 2, 3, 4, 5 or greater than 5 amino acid alterations.

A derivative of a peptide for which the specific sequence is disclosed herein may be in certain embodiments the same length or shorter than the specific peptide. In other embodiments, the peptide sequence or a variant thereof may include a larger peptide.

As is well understood, homology at the amino acid level is generally in terms of amino acid similarity or identity. Similarity allows for ‘conservative variation’, such as substitution of one hydrophobic residue such as isoleucine, valine, leucine or methionine for another, or the substitution of one polar residue for another, such as lysine, glutamic acid for aspartic acid, or glutamine for asparagine.

Analogues of, and for use in, the invention as defined herein means a peptide modified by varying the amino acid sequence e.g. by manipulation of the nucleic acid encoding the protein or by altering the protein itself. Such derivatives of the amino acid sequence may involve insertion, addition, deletion and/or substitution of one or more amino acids, while providing a peptide capable of inducing modulating the activity of a lactating cell, either on its own, or in combination with other peptides.

Analogues also include derivatives of the defined peptides, including the peptide being linked to a coupling partner, e.g. an effector molecule, a label, a drug, a toxin and/or a carrier or transport molecule, or a transport molecule. Techniques for coupling the peptides of the invention to both peptidyl and non-peptidyl coupling partners are well known in the art.

Non-peptide mimetics are further provided within the scope of the invention. Peptides according to the invention may be prepared, either wholly or partly, by chemical synthesis. Generation of the peptides in this way can be performed by methods which are well known to the person skilled in the art of the present invention, particularly standard liquid or solid-phase peptide synthesis methods. Another way of producing the peptides according to the invention is to express the nucleic acid encoding the amino acid sequences in a nucleic acid expression system.

According to a second aspect of the present invention, there is provided a peptide including the amino acid sequence of SEQ ID NO:1, SEQ ID NO:2 or SEQ ID NO:3, which in combination with one or more further peptides including the amino acid sequence shown SEQ ID NO:1, SEQ ID NO:2 or SEQ ID NO:3 provides a reduction in milk secretion from a lactating cell.

In one preferred embodiment, the peptide includes the amino acid sequence shown in SEQ ID NO:1 or an analogue thereof.

In another preferred embodiment, the peptide includes the amino acid sequence shown in SEQ ID NO:2 or an analogue thereof.

In a yet further preferred embodiment, the peptide includes the amino acid sequence shown in SEQ ID NO:3 or an analogue thereof.

Preferably the peptide has a molecular mass determined by mass spectrometric analysis of between 1000 to 3000 Da.

In a particular embodiment of the invention, the peptide is glycosylated.

Alternatively the peptide is unglycosylated.

Further, the peptides of the present invention can be in either phosphorylated or unphosphorylated form.

The present invention further includes truncated versions of the peptides which have been isolated from milk, and in particular cow's milk.

Analogues of and for use in the invention as defined herein means a peptide modified by varying the amino acid sequence e.g. by manipulation of the nucleic acid encoding the protein or by altering the protein itself. Such derivatives of the amino acid sequence may involve insertion, addition, deletion and/or substitution of one or more amino acids, while providing a peptide capable of influencing milk secretion either on its own, or in combination with other peptides.

Preferably such analogues involve the insertion, addition, deletion and/or substitution of 5 or fewer amino acids, more preferably of 3 or fewer, and most preferably of only 1 or 2 amino acids.

Analogues also include derivatives of the defined peptides, including the peptide being linked to a coupling partner, e.g. an effector molecule, a label, a drug, a toxin and/or a carrier, targeting or transport molecule. Techniques for coupling the peptides of the invention to both peptidyl and non-peptidyl coupling partners are well known in the art.

A further aspect of the invention provides a peptide mixture comprising two or more different peptides, the peptides including the amino acid sequence of SEQ ID NO:1, SEQ ID NO:2 or SEQ ID NO:3, or analogues thereof.

A yet further aspect of the present invention provides a method of influencing milk secretion in animals, the method including the steps of administering at least one peptide according to the invention to a lactating animal.

Preferably the term animal is taken to include humans.

In one preferred embodiment, the animal is a cow, goat or sheep.

Suitable methods of administration will be known to the person skilled in the art. Such methods are discussed under Example 7 of the present invention and include intravenous, intra-mammary, subcutaneous, and intra-ductal through the teat into the mammary milk storage space.

A yet further aspect of the present invention provides for antibodies directed to a peptide including the amino acid sequence shown in SEQ ID NO:1, SEQ ID NO:2 or SEQ ID NO:3.

Preferably said antibodies promote or improve lactation in humans and other mammals.

Preferably said antibodies modulate the milk secretion rate of lactating cells through the promotion or improvement of lactation in animals, most preferably humans, sheep, cows and goats. Without wishing to be bound by theory, it is believed that the antibodies act through inhibiting the action of peptides which include the amino acid sequence shown in SEQ ID NO:1, SEQ ID NO:2 or SEQ ID NO:3.

An “antibody” is an immunoglobulin, whether natural or partly or wholly synthetically produced. The term also covers any polypeptide, protein or peptide having a binding domain which is, or is homologous to, an antibody binding domain. These can be derived from natural sources, or they may be partly or wholly synthetically produced. Examples of antibodies are the immunoglobulin isotypes and their isotypic subclasses and fragments which comprise an antigen binding domain such as Fab, scFv, Fv, dAb, Fd, and diabodies.

The binding member of the invention may be an antibody such as a monoclonal or polyclonal antibody, or a fragment thereof. The constant region of the antibody may be of any class including, but not limited to, human classes IgG, IgA, IgM, IgD and IgE. The antibody may belong to any sub class e.g. IgG1, IgG2, IgG3 and IgG4.

As antibodies can be modified in a number of ways, the term “antibody” should be construed as covering any binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023.

It has been shown that fragments of a whole antibody can perform the function of antigen binding.

Examples of such binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341:544-546 (1989)) which consists of a VH domain; (v) isolated CDR regions; (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., Science 242:423-426 (1988); Huston et al., PNAS USA 85:5879-5883 (1988)); (viii) bispecific single chain Fv diners (PCT/US92/09965) and (ix) “diabodies”, multivalent or multispecific fragments constructed by gene fusion (WO94/13804; P. Hollinger et al., Proc. Natl. Acad. Sci. USA 90:6444-6448 (1993)).

The term “antibody” includes antibodies which have been “humanised”. Methods for making humanised antibodies are known in the art. Methods are described, for example, in Winter, U.S. Pat. No. 5,225,539. A humanised antibody may be a modified antibody having the hypervariable region of a monoclonal antibody such as 791T/36 and the constant region of a human antibody. Thus the binding member may comprise a human constant region.

In an alternative embodiment, inhibition of the action of a peptide or peptides including the amino acid sequence shown in SEQ ID NO:1, SEQ ID NO:2 or SEQ ID NO:3 can be effected by a small molecule which inhibits the biological function of the peptide, or further by down regulation of a gene encoding for a peptide or more generally for genes encoding for the peptides. Assay methods for assessing the effectiveness of small molecules as inhibitors of the peptides of the present invention are also within the scope of the present invention.

A yet further aspect of the present invention provides a composition for influencing lactation in animals, the composition including a peptide including the amino acid sequence RPKHPIKHQG (SEQ ID NO:1), AVAVSQEAN (SEQ ID NO:2) or SEGVALDPAR (SEQ ID NO:3) or an analogue thereof.

Preferably the animal is a non-human animal.

More preferably the animal is a cow, goat or sheep.

Preferably the composition inhibits lactation in the target cells within hours of administration, with the response dependent on the frequency of milk removal from the mammary gland.

Preferably the composition is administered by intra-ductal injection into the mammary gland at a dose level yielding a final concentration of peptides in milk in the range 0.1 to 16 micromolar. The administration of this dose may be repeated as required, and possibly increased when given over long periods.

A yet further aspect of the present invention provides a method of modulating the milk secretion rate of a lactating cell, the method including the steps of;
selecting a composition including at least one peptide including the amino acid sequence RPKHPIKHQG (SEQ ID NO:1), AVAVSQEAN (SEQ ID NO:2) or SEGVALDPAR (SEQ ID NO:3) or an analogue thereof,
administering the composition to the animal through the local targeting of the mammary gland, and
exposing the cells of the mammary gland to a concentration of the composition sufficient to induce a biochemical feedback mechanism and reduce lactation by the mammary cells.

Preferably this method is used in farming to provide an improvement in husbandry practices in order to achieve higher levels of lactation and product yield.

Preferably the composition is administered through intra-ductal administration.

Preferably delivery of the composition is by means of a bolus of peptide which is preferably encapsulated. Preferably the encapsulation material is a non-phosphate containing isotonic buffer at the physiological pH of milk i.e. pH 6.7.

Preferably the composition is administered at a dose yielding a final concentration of peptides in milk in the range 0.01-1.6 micromolar.

Alternatively the composition is administered at a dose yielding a final concentration of peptides in milk in the range 10-16 micromolar.

Further, the composition is administered at a dose yielding a final concentration of peptides in milk in the range 0.01-16 micromolar.

Without wishing to be bound by theory, it is theorised that the reduction in milk production by lactating cells is caused by modulation by the peptides of the invention of the lactation promoting effects of galactopoietic hormones. In particular, these hormones influence the control of gene expression in the mammary cell.

A yet further aspect of the present invention provides for the use of at least one peptide including the amino acid sequence RPKHPIKHQG (SEQ ID NO:1), AVAVSQEAN (SEQ ID NO:2) or SEGVALDPAR (SEQ ID NO:3) or an analogue thereof in the preparation of a composition for the modulation of lactation in a lactating cell of an animal.

According to a yet further aspect of the present invention there is provided a method of treating mastitis, the method including the step of administering a therapeutically useful amount of a at least one peptide including the amino acid sequence RPKHPIKHQG (SEQ ID NO:1), AVAVSQEAN (SEQ ID NO:2) or SEGVALDPAR (SEQ ID NO:3) or an analogue thereof.

In one preferred embodiment, the method of treatment can be used to treat infection of the teat or mammary gland, the method including the step of administering a therapeutically useful amount of at least one peptide including the amino acid sequence RPKHPIKHQG (SEQ ID NO:1), AVAVSQEAN (SEQ ID NO:2) or SEGVALDPAR (SEQ ID NO:3) or an analogue thereof to a subject sufficient to cause protection against infection.

A yet further aspect of the present invention provides the use of a peptide including the amino acid sequence shown in SEQ ID NO:1, SEQ ID NO:2 or SEQ ID NO:3 in the preparation of a medicament for the treatment of mastitis.

The term ‘treatment’ as used herein refers to any regime that can benefit a human or non-human animal.

The treatment may be in respect of an existing condition or may be prophylactic (preventative treatment). Treatment may include curative, alleviation or prophylactic effects.

In a yet further aspect of the present invention, there is provided use of at least one peptide including the amino acid sequence shown in SEQ ID NO:1, SEQ ID NO:2 or SEQ ID NO:3 or analogues thereof in the elucidation of a negative feedback pathway, in order to restrict lactation in an animal model.

In a still further aspect of the present invention there is provided a kit for the performance of any one of the assay methods of the invention, said kit comprising at least one peptide according to the invention together with instructions and protocols for the performance of the method(s).

Preferred features of each aspect of the invention are as for each of the other aspects mutatis mutandis unless the context demands otherwise.

Unless otherwise defined, all technical and scientific terms used herein have the meaning commonly understood by a person who is skilled in the art in the field of the present invention.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

In summary, the present invention provides the surprising and unexpected finding that at least one peptide which can act in combination with other defined peptides, inhibits the secretion of milk constituents in primary cell cultures that reproduce the activities of lactating mammary tissue.

### BRIEF DESCRIPTION OF THE DRAWINGS

The present invention will now be described, by way of example only, with reference to the accompanying drawings, wherein;

FIG. 1 shows the resolution of the 6-30 kDa whey fraction by Sephacryl gel filtration chromatography,

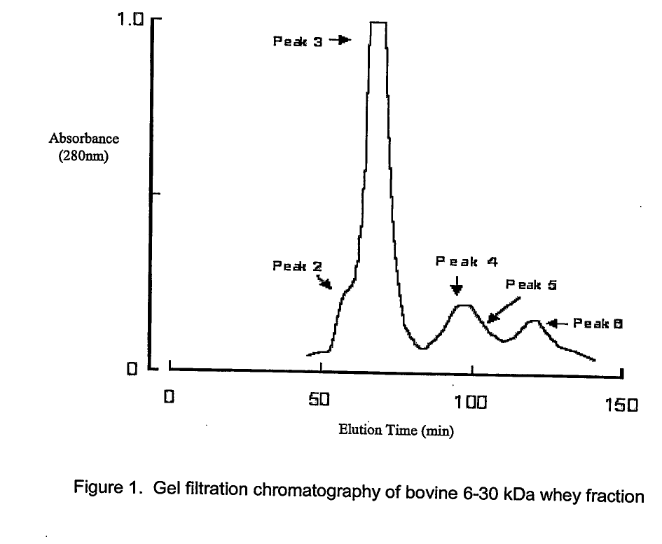

FIG. 2 shows the further resolution of gel-filtration peak S4 by Superdex peptide chromatography,

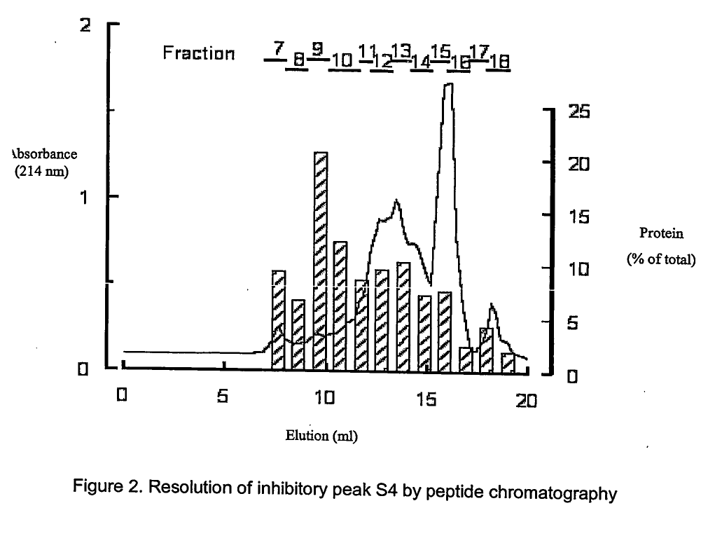

FIG. 3 shows an example of the further resolution of the Superdex fraction P8-11A by reversed phase HPLC,

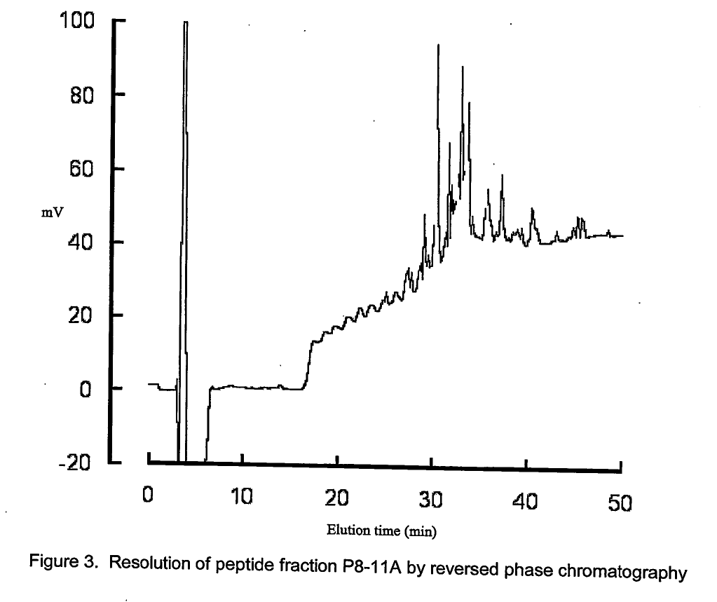

FIG. 4 shows the inhibition of protein secretion in acini cultures by fractions obtained through resolution of a 6-30 kDa fraction of cow's whey proteins by Sephacryl gel filtration chromatography,

FIG. 5 shows the inhibition of protein secretion in cell culture by fractions of prepared by Superdex high-resolution peptide chromatography,

FIG. 6 shows inhibition of protein secretion in mammary acini cultures by components of peptide fraction 8-11A resolved by reversed phase HPLC,

FIG. 7 shows the effect of peptide A on protein secretion in mammary cell cultures,

FIG. 8 shows the effect of peptide B on protein secretion in mammary cell cultures,

FIG. 9 shows the effect of peptide C on protein secretion in mammary cell cultures,

FIG. 10 shows the effect of a combination of peptides A, B and C on protein secretion in mammary cell cultures, and

FIG. 11 shows the effect of triple peptide treatment for 2 days on milk protein mRNA abundance in bovine mammary epithelial cells in culture.

### DETAILED DESCRIPTION OF THE INVENTION

Without wishing to be bound by theory, the inventors predict that the peptides of the present invention may exist in cow's milk; possibly in glycosylated or phosphorylated form. The peptides may act together to inhibit the rate of milk secretion in the mammary gland.

The peptides of the invention can be obtained from cow's milk by a method described herein or by some variant thereon. It has been demonstrated that the three peptides of the invention isolated from cow's milk are able to inhibit the secretion of milk proteins in mammary acini cultures. When the three peptides are present together in a milk fraction added to the culture medium for a two hour period, they are able to inhibit protein secretion in a concentration-dependent manner.

Synthetic peptides based on the N-terminal sequence of the natural peptides can be synthesised by standard Fmoc amino acid chemistry. Synthetic peptides produced according to the N-terminal sequence of the peptides of the invention, and representing truncated 9- or 10-amino acid forms of the natural peptides similarly inhibit the secretion of protein in primary cultures of mammary cells prepared as acini by collagenase digestion of lactating tissue. Inhibition is exerted acutely, within two hours, and is elicited in a concentration-dependent manner by synthetic peptides in combination. It is expected that the inhibitory activity of synthetic peptides will depend on the proportion of the full-length sequence synthesised and that the inhibitory potency of these and the natural peptides will depend on the degree of peptide modification by glycosylation or phosphorylation.

The invention is applicable to any animal responsive to the inhibitory peptides defined herein. In addition, the demand-led relationship between milk supply and milk removal in most if not all mammals predicts that the same effects will be demonstrable in relation to the peptides of the invention obtained from milk of other species, in relation to that species.

In man, administration by a suitable route of the peptides, or antibodies thereto, may be applied to improve or suppress lactation. In dairy cows, there may be a need to reduce milk yield in order to maintain production within quota limits, in which case the inhibitory peptides themselves are administered. For intra-ductal injection of peptides into the mammary gland, a dose yielding a final concentration of peptides in milk in the range 0.01-1.0 micromolar is likely to be effective, and should be repeated as required, and possibly increased when given over long periods.

Conversely, passive immunisation methods using antibodies against the peptides may be used to generate a reduction in the effect of the natural inhibitory peptides when this is desired in order to increase milk supply in lactating animals.

Antibodies against the natural peptides of the invention or against their synthetic analogues can be raised by conventional methods such as polyclonal antisera, mouse monoclonal antibodies, cow-mouse hybrid monoclonal antibodies or as engineered antibodies by any of the currently available methods known to the skilled man in the field. Conventional carriers and adjuvants known in vaccination can be used. Antibodies against synthetic truncated peptides based on the sequence of the peptides of the invention may be used to isolate the natural peptides from cow's milk extracts, or to control milk supply as described above.

### EXAMPLES

### Example 1

Preparation of Cow Milk Fractions

Milk was collected at the morning milking from Friesian cows, and was defatted by centrifugation (2500 g, 15° C., 20 minutes) and filtered through glass wool. Casein in defatted milk was precipitated by dropwise addition of concentrated HCl until the pH reached 4.6. After stirring for 10 minutes, casein was sedimented by centrifugation (2500 g, 15° C., 20 minutes), and the clear whey supernatant was filtered through glass fibre membranes of decreasing pore size, the final membrane made of polyethersulphone having a cut-off of 0.45 microns.

The whey fraction was subjected to ultrafiltration using a filter with a nominal cut-off value of molecular weight 30,000 Daltons(Da). The filtrate was dialysed for 24 hours against 10 mM sodium acetate buffer pH 4.6 containing 1.5 mM ε-aminocaproic acid, 100 μM glutathione, 1 mM EDTA and 1 mM EGTA using a dialysis membrane with a nominal molecular weight cut-off of 6,000 Da, and was then adjusted to pH 7.0 by addition of NaOH. The neutralised filtrate was dialysed against 2 mM phosphate buffer pH 7.0 containing 1.5 mM ε-aminocaproic acid, 100 μM glutathione, 1 mM EDTA and 1 mM EGTA for 24 hours and then freeze dried.

### Example 2

Gel Filtration Chromatography

The 6-30 kDa whey fraction was resolved of a Hi-Prep Sephacryl S-100 high resolution gel filtration column (Pharmacia) using a Fast Protein Liquid Chromatography (FPLC) system (Pharmacia). The freeze-dried whey fraction was reconstituted in one tenth its volume before freeze-drying, and the solution was clarified by filtration through a 0.22 μm filter. The chromatography buffer was 20 mM phosphate buffer pH 7.0 containing 0.15 M NaCl, and was filtered through a 0.22 μm filter and degassed before use. 2 ml of the 10× concentrated whey fraction was loaded for each separation. The flow was 1 ml/min.

Fractions containing protein peaks eluted from the column were tested for inhibitory activity in a cell culture bioassay (see Example 3 below), and fractions spanning one protein peak containing inhibitory activity, designated peak S4, were combined and desalted by passage through a column composed of Poros 50 R2, composed of cross-linked poly(styrene/divinylbenzene) (PerSeptive Biosystems). Protein bound to the column was washed with distilled water, and then eluted with 80% (v/v) acetonitrile in distilled water. The inhibitory fraction was then concentrated under a stream of nitrogen and freeze dried. A second peak of inhibitory material, designated peak S6, contained too little protein for further purification to be practicable.

Fraction S4 was resolved further on a Superdex Peptide HR 10/30 column (Pharmacia) using an FPLC system. Dried fraction was reconstituted in 0.2-0.4 ml of solution of 0.2 mM AEBSF, 10 mM EDTA and 10 mM EGTA, and chromatography was performed in 20 mM phosphate buffer pH 7.0 containing 0.25 M NaCl at a flow rate of 1 ml/min. Fractions containing protein peaks eluting from the column were collected. These were either desalted on a Poros column and freeze dried as described above for assay of inhibitory activity, or were freeze dried immediately for further fractionation. Inhibitory material was detected in the leading edge of the major peak, designated fraction P8-11A.

Fraction P8-11A was resolved further using a reversed phase HPLC column (Genesis 25 cm C18, 4 microns: Jones chromatography) on a Spectra Physics HPLC system. Sample containing the equivalent of one Superdex fraction P8-11A was dissolved in water and loaded in a volume of 0.2 ml. The column was eluted with a gradient of 0-60% acetonitrile in 0.1% (w/v) trifluoroacetic acid (TFA), and 1 ml fractions of the eluate were collected. HPLC fractions were freeze dried, and tested for inhibitory activity as described above.

### Example 3

Mammary cell culture bioassay of milk fractions

Mammary cells were prepared from tissue of lactating mice by collagenase digestion according to the method of K Hendry, K Simpson, K Nicholas & C Wilde. Journal of Molecular Endocrinology 21: 169-177 (1998). The resultant suspension of mammary acini consisted predominantly of groups of 50-200 cells, and was cultured in medium (Medium 199/Ham's F12: 50:50 v/v) containing insulin (5 μg/ml), hydrocortisone (0.1 μg/ml) and prolactin (1 μg/ml). Culture density was 1.5×10⁶ cells/ml, and cells were maintained at 37° C. in an atmosphere of 5% CO₂ in air. Protein synthesis and secretion were measured by continuous labelling with L-[4.5-3H]leucine (40-70 μCi/mmol; 10-20 μCi/ml) for 2 hours in the presence or absence of milk fractions (at concentrations of 0.2-4.0 μg/ml) or synthetic peptides (0.01-10 μM). Milk extracts or synthetic peptides were dissolved and diluted in 10 mM Hepes buffer pH 7.4, and control cultures containing only diluent were included in each experiment. The culture was terminated by centrifugation of the cell suspension (2000 g, 2 minutes), and the cell pellet and supernatant were frozen and stored separately for assay of DNA and protein secretion respectively. Radiolabel incorporation was measured by precipitation with trichloroacetic acid (final concentration 10% w/v). Cell lysates were prepared by sonication using a Kontes KT50 cell disrupter (setting 20, 15 s) in 0.1 M NaH₂PO₄ pH 7.4 containing 2 M NaCl, and assayed for DNA content by the method of C Labarca and K Paigen, Analytical Biochemistry 102: 344-352 (1980). Secretory activity was expressed per unit of cellular DNA.

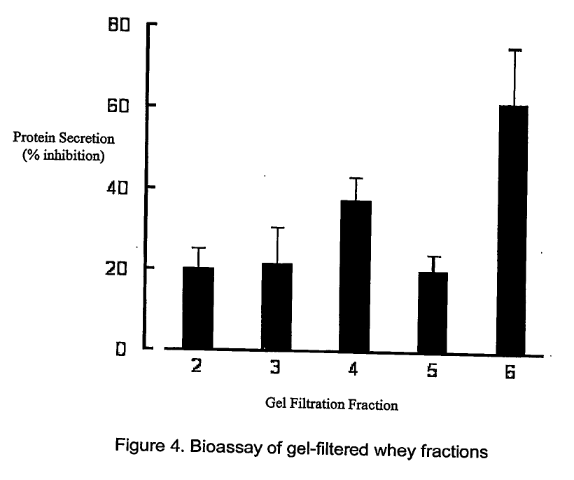

The amount of radiolabelled protein secreted by acini in the presence of milk extracts or synthetic peptides was expressed as a percentage of that produced by the cells to which no milk fraction or peptide was added. In each experiment, treatments were replicated in a minimum of three culture wells, and results for individual experiments were mean values for those wells. Values shown in FIGS. 4 to 6 are the mean for three or four experiments, each testing a different preparation of milk fractions. Error bars show the standard error of the mean for these experiments.

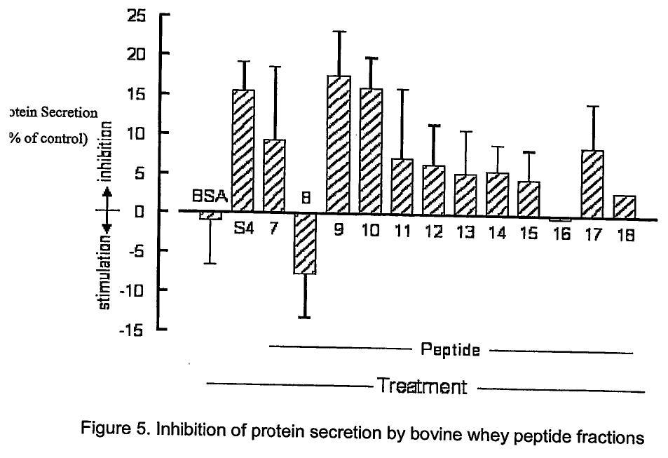

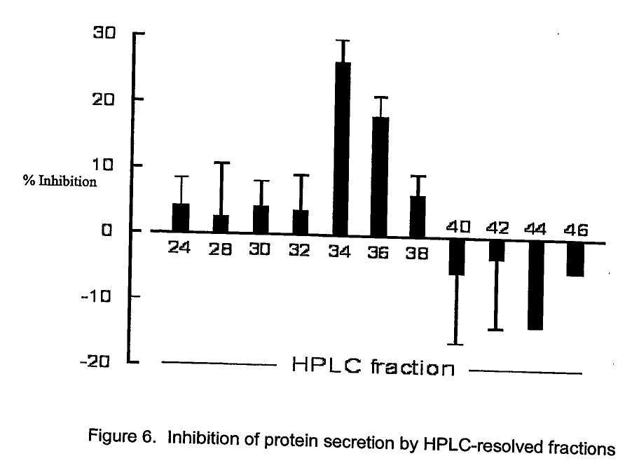

FIG. 4 shows the inhibition of protein secretion in acini cultures by fractions obtained through resolution of a 6-30 kDa fraction of cow's whey proteins by Sephacryl gel filtration chromatography. FIG. 5 shows the inhibition of protein secretion in cell culture by fractions prepared by Superdex high-resolution peptide chromatography. FIG. 6 shows inhibition of protein secretion in mammary acini cultures by components of peptide fraction 8-11A resolved by reversed phase HPLC.

### Example 4

Preparation of Synthetic Peptides Based on Natural Peptide Sequences

N-terminal sequencing of the inhibitory HPLC fraction eluting at 36-39% acetonitrile revealed three peptides (Peptides A, B and C) with the N-terminal sequences:

| | |
|---|---|
| Peptide A: | RPKHPIKHQG |
| Peptide B: | AVAVSQEAN |
| Peptide C: | SEGVALDPAR |

Peptide A (SED ID NO:1) was identified as the N-terminal sequence of αₛ₁-casein. Peptides B (SED ID NO:2) and C (SED ID NO:3) have limited homology to known amino acid sequence data (Swiss-Prot protein sequence database: URL—www.swissprot.com). Mass spectrometric analysis indicated that peptides had masses in the range 1000-3000 Daltons.

Peptide A was produced synthetically by chymosin digestion of αₛ₁′-casein purified by a modification of the method of E. Lahov and W. Regelson, Food Chemistry and Toxicology 34:1 131-145 1996. The N-terminal fragment of mass 2762 Da was isolated from the chymosin digest by reversed phase chromatography system. This peptide consists of a 23 amino acid sequence at the N-terminus of the αₛ₁-casein protein.

N-terminal sequences of peptides B (SEQ ID NO:2) and C (SEQ ID NO:3) comprising nine and ten amino acids respectively were synthesised by solid phase synthesis using Fmoc amino acids coupled by the PyBOP/HOBt/DIPEA method. The peptides were cleaved from the resin with 80% TFA plus suitable scavengers and purified by reverse phase HPLC on a Phenomenex Luna 10μ C18 column of dimensions 25 cm×0.212 cm using a linear gradient of water to 100% acetonitrile in 0.1% TFA.

### Example 5

Mammary Culture Bioassay of Synthetic Peptides

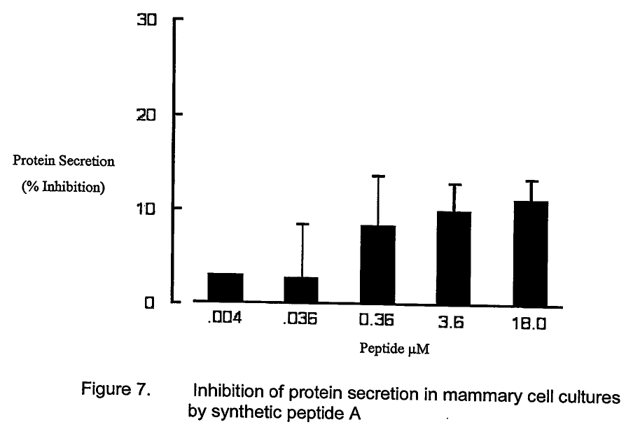

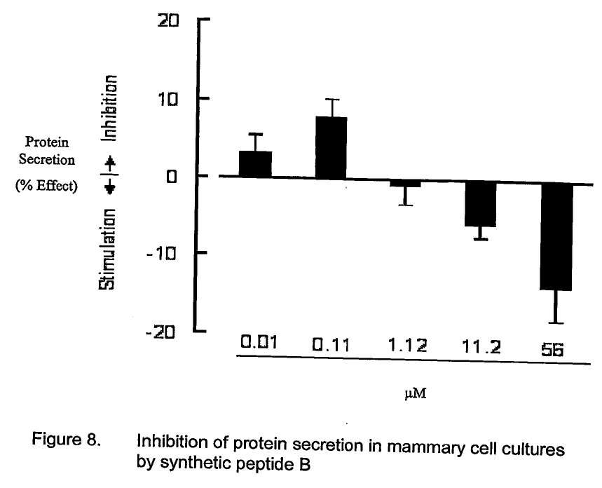

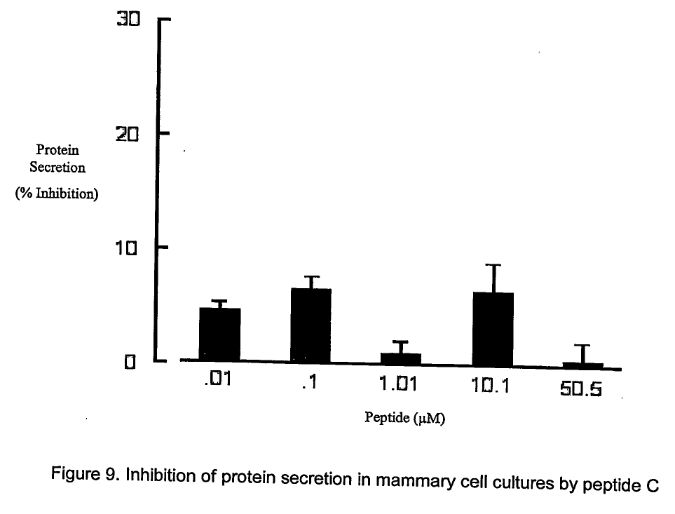

Protein secretion was measured in mammary acini cultures in the presence and absence of synthetic peptides. Peptides were added singly or in combination at equimolar concentrations over a range of 0.01-10.0 μM. In each experiment, treatments were replicated in a minimum of three culture wells, and results for individual experiments were mean values for those wells. Values shown in FIGS. 7 to 9 are the mean for three experiments. Error bars show the standard error of the mean for these experiments.

Statistical analysis of bioassay data showed that, together, the three peptides exhibited a concentration-dependent inhibition of secretion. Maximal inhibition of secretion was obtained at a concentration of 0.1-1.0 μM of each peptide. None of the peptides tested individually inhibited secretion to this extent, and at higher concentrations the inhibitory action of peptide A was counteracted by a stimulatory effect of peptide B. The effects of the synthetic peptides indicate that inhibition of secretion by the HPLC fraction containing natural peptides with the same N-terminal sequences is due to the combined actions of the peptides, and is unlikely to be conferred by any one constituent of the active HPLC fraction.

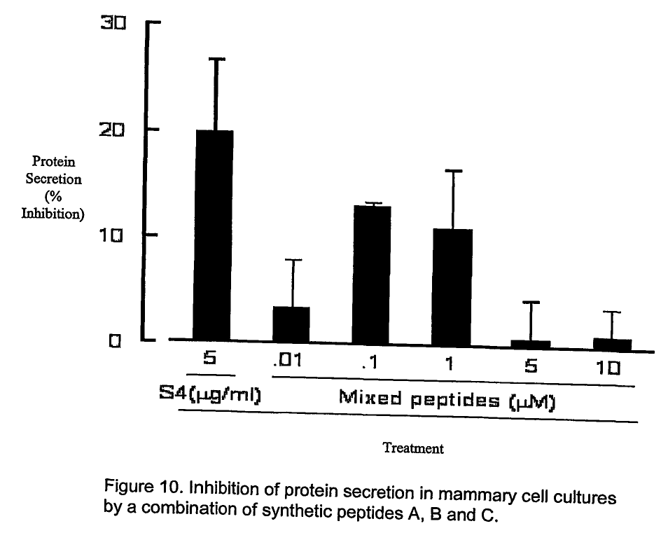

FIGS. 7, 8 and 9 show the effect of peptides A, B and C on protein secretion in mammary cell cultures. FIG. 10 shows the effect of a combination of peptides A, B and C on protein secretion in mammary cell cultures.

### Example 6

The inhibition of mammary cell function were described initially in short term (acini) cultures over a period of 2 hours (Example 3). The effects were unlikely to be exerted through modulation of gene expression. It was therefore of interest to ascertain if, in addition, the peptides also influenced mammary mRNA abundance, an index of the degree of cellular differentiation. If so, this would implicate the peptides in mediation of a second phase of the response of milk secretion to a change in milk removal: short term up- or down-regulation of milk yield by a change in milking frequency is, if the stimulus is sustained, followed in the medium term by modulation of the abundance of important mammary mRNA levels.

Method

A novel culture system was developed to allow the study of bovine mammary epithelial cells in vitro, under conditions where the cells were differentiated and exhibited functions exclusive to lactating mammary tissue.

Essentially, this was achieved by culturing cells on a reconstituted basement membrane extracted from the murine Engelbreth-Holm-Swarm sarcoma (termed EHS matrix) and in the presence of lactogenic hormones. It is well-established using mammary cells of non-ruminant species, that epithelial cells cultured on this substratum acquire the architecture and function of cells in lactating tissue. This proved also to be the case with bovine mammary epithelial cells, which expressed milk protein genes indicative of differentiated i.e. lactating function, and secreted the milk protein gene products.

The peptides tested were based on the structures of those determined previously to inhibit secretion in mammary acini cultures. Peptide A (SEQ ID NO:1) was used as a 15-residue peptide based on the published N-terminal structure of the native protein (Lahov E & Regelson W. Food Chemistry and toxicology 34: 131-145), at concentrations of 1.6 μM and 16 μM.

Peptide B was tested at concentrations of 1 μM and 10 μM as a 10-residue molecule comprising the amino acid sequence determined previously plus a C-terminal cysteine residue. Peptide C was tested at concentrations of 1 μM and 10 μM as a 10 amino acid residue peptide, according to the sequence determined previously.

In one experiment both peptides A and C were modified to contain a C-terminal cysteine residue, generating 16-residue and 11-residue peptides respectively. Cysteine elongation was included so that the cell-biological effect of this modification, introduced during antibody generation, could be assessed.

Throughout, peptides were tested in combination, mixing peptide A (1.6 μM) with peptides B and C (1 μM each), or mixing peptide A (16 μM) with peptides B and C (10 μM each).

Mammary cell function was measured in terms of milk protein mRNA abundance, using P-casein and lactoferrin as indicators of differentiated function. In each case, milk protein mRNA abundance was measured by quantitative PCR, using standard methods. Results were standardized between experiments by expressing data relative to glyceraldehyde-3-phosphate dehydrogenase (GAPDH) mRNA, generally accepted to be a housekeeping gene whose abundance should be unaffected by the treatment.

Results

(i) Milk Protein Gene Expression and Protein Secretion

Four experiments were performed, each with a different pool of bovine mammary epithelial cells.

In each experiment, cells expressed P-casein and lactoferrin, although the level of expression differed between cell-pools.

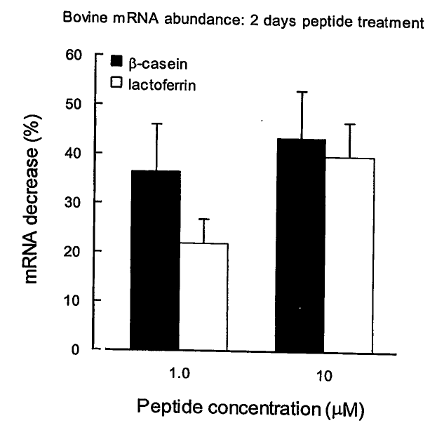

Peptide treatment consistently decreased the abundance of mRNAs for β-casein, and had a similar effect on the abundance of lactoferrin mRNA (FIG. 11), when each was expressed relative to GAPDH mRNA. In the case of β-casein, message abundance decreased by 43% after two days treatment with 10 μM concentrations of the peptides. The effect on lactoferrin was equally marked, with an average reduction in mRNA level of 35%. Cysteine elongation of peptides A and C in one experiment did not obviate the peptides' ability to decrease milk protein mRNA abundance in culture.

A similar reduction was observed in the secretion of casein. Peptide concentrations of 1 μM decreased total casein secretion by 13% after two days of treatment, and concentrations of 10 μM reduced casein secretion by 26%. These results together suggest that the three peptides in combination inhibit differentiated function in the mammary epithelial cells, an effect that, when elicited in vivo, would result in a sustained decrease in milk production or, potentially, switching off of lactation.

The decrease in milk protein mRNA abundance was in part accounted for by an unexpected increase in the level of GAPDH mRNA abundance, against which the milk protein measurements were standardized. Compared with controls, GAPDH mRNA abundance in cells treated with 1 μM and 10 μM peptides increased 2.6-fold and 1.5-fold respectively. Use of a nominally housekeeping gene is common practice in presentation of mRNA data obtained by PCR, as it controls for experimental variation in the number of cells contributing to the measurements. GAPDH and α-actin are amongst those often used as housekeeping genes; as α-actin mRNA abundance is very low in bovine mammary epithelial cell cultures, GAPDH was chosen.

(ii) Expressing Data Relative to glyceraldehyde-3-phosphate dehydrogenase (GAPDH) mRNA, generally Accepted to be a Housekeeping Gene

An effect of triple peptide treatment on GAPDH mRNA abundance was unexpected, and indicates that this gene cannot be considered a suitable control in measurement of mammary gene expression. This conclusion is supported by recent microarray study of mammary gene expression, which reports that GAPDH mRNA abundance changes in the course of a lactation cycle in the mouse (Clarkson et al. (2004) Breast Cancer Research 6: R92-R109).

An explanation for the peptides' increase in GAPDH mRNA level is currently under investigation.

However, information is accumulating that, in addition to its function as an intrinsic metabolic enzyme, GAPDH may be involved in intra-cellular vesicle trafficking, that this activity is subject to control by known signalling pathways, and that modulation of those signals can alter GAPDH phosphorylation status and impede vesicle movement and therefore secretory pathways (Tisdale E J. (2002) Journal of Biological Chemistry 277:3334-41). Therefore, it may be that, rather than being simply an unsuitable control marker of cellular gene expression, the peptides' effect on GAPDH may be important for their inhibition of mammary secretory function. Acute inhibition of secretion and medium-term reductions in milk protein mRNAs may then be a consequence of an effect on GAPDH. Recently, we have shown that peptide treatment of bovine mammary epithelial cells for two days using 1 μM or 10 μM of each of the three peptides, also increased GAPDH protein abundance. We are investigating the possibility that this cellular response is to counter-act an inhibition of GAPDH trafficking activity through alteration in the protein's phosphorylation status.

### SUMMARY

The combination of three peptides (A, B and C), when used to treat bovine mammary epithelial cells in primary culture, caused a decrease in the abundance of milk protein mRNAs. This confirms the peptides' ability to inhibit activities associated with milk secretion in the lactating mammary cell

### Example 7

In-Vivo Animal Model Procedures

(i) Small Animal Model

A suitable small animal model to demonstrate the regulation of milk secretion from lactating cells is the lactating mouse. In this example, the animals are preferably in mid-lactation, 8 to 10 days after parturition, and are suckling a litter of at least 8 pups. The peptide treatment is administered by injection into the inguinal (lower abdominal) mammary gland on one side of the body. The peptides are encapsulated in an oil-based formulation, ideally a mineral oil. Treatment is for up to 24 hours for measurement of milk constituents, or for 24 hours or multiples thereof when milk protein mRNA abundance is measured. The effect of the peptide or peptides on milk constituent levels in the mammary gland, over 4 to 24 hours, is measured after removal of the litter, or sealing of the teats with tissue adhesive, to prevent milk removal. The effect of the peptides on milk protein mRNA levels measured after 24 hours or multiples thereof is measured without prevention of litter removal. In both cases, measurements in the treated gland are compared with those in the equivalent contra-lateral gland which would be treated simultaneously with only the encapsulation material (i.e. a control preparation).

(ii) Large Animal Model

A suitable large animal model is a lactating dairy cow, in which the preferred route of administration of a peptide or peptides is as described above, or analogues or antibodies thereof, is by intra-ductal injection into the mammary gland. Peptides given by intra-ductal injection are administered in a small volume (usually 10-50 ml) of isosmotic solution at the physiological pH of milk, normally pH 6.6-6.8. Preferably, treatment is given after the afternoon milking in cows milked twice daily, and may be repeated on one or more consecutive days. Treatment is preferably restricted to one quarter of the udder, and one or more other quarters receive the excipient solution only. The effect of treatment is assessed by calculating the daily milk yield of the treated quarter before and after treatment. This change is then compared with that in control glands receiving excipient only over the same period, allowing each animal to be used as its own control. In addition to milk yield measurement, needle biopsy of treated and control mammary glands may be used to compare the effect of peptide treatment on the abundance in mammary tissue of milk protein mRNAs.

Pharmaceutical Compositions

As described above, the present invention extends to a pharmaceutical composition for the regulation of milk secretion from a lactating cell, wherein the composition comprises at least one peptide having the sequence defined in SEQ ID NO:1, SEQ ID NO:2, or SEQ ID NO:3. Pharmaceutical compositions according to the present invention, and for use in accordance with the present invention may comprise, in addition to active ingredient (i.e. one or more peptides), a pharmaceutically acceptable excipient, carrier, buffer stabiliser or other materials well known to those skilled in the art. Such materials should be non-toxic and should not interfere with the efficacy of the active ingredient. The precise nature of the carrier or other material will depend on the route of administration, which may be, for example intravenous, sub-cutaneous, intra-mammary, or intra-ductal through the teat into the mammary milk-storage space.

The formulation may be a liquid, for example, a physiologic salt solution containing non-phosphate buffer at pH 6.7 to 7.6 when the peptides are administered systemically or into the mammary teat duct. For intra-mammary treatment by injection into the gland, the preferred formulation is an oil-based formulation, preferably using mineral oil.

Dose

The composition is preferably administered to an individual in a “therapeutically effective amount”, this being sufficient to show benefit to the individual. The actual amount administered, and rate and time-course of administration, will depend on the nature of the animal being treated.

The optimal dose can be determined based on a number of parameters including, for example animal type, age and stage through lactation, the precise form of the composition being administered and the route of administration.

The composition may be administered via microspheres, liposomes, other microparticulate delivery systems or sustained release formulations placed in certain tissues including blood. Suitable examples of sustained release carriers include semipermeable polymer matrices in the form of shared articles, e.g. suppositories or microcapsules. Implantable or microcapsular sustained release matrices include polylactides (U.S. Pat. No. 3,773,919 and European Patent Application Publication No 0,058,481) copolymers of L-glutamic acid and gamma ethyl-L-glutamate (Sidman et al., Biopolymers 22(1): 547-556, 1985), poly (2-hydroxyethyl-methacrylate) or ethylene vinyl acetate (Langer et al., J. Biomed. Mater. Res. 15: 167-277, 1981, and Langer, Chem. Tech. 12:98-105, 1982, the entire disclosures of which are herein incorporated by reference).

Examples of the techniques and protocols mentioned above and other techniques and protocols which may be used in accordance with the invention can be found in Remington's Pharmaceutical Sciences, 16th edition, Oslo, A. (ed), 1980, the entire disclosure of which is herein incorporated by reference.

### Example 8

Utility in Human Breast Feeding

The present invention would have particular utility in the regulation of human breast-feeding. In addition to the stimulus provided by a suckling new born infant, antibodies directed to peptides according to the invention can be suitably administered, this resulting in the upregulation of milk production. This upregulation in production of milk by mammary cells can be particularly advantageous, particularly early on during the breast feeding period, where there may be insufficient natural levels of lactation by the mother in order to satisfy the nourishment requirements of the new born child.

The promotion of lactation in the mother can also be important when feeding premature babies as the lactation inducing signals will not be at a high level. However, the ability of the mother to lactate and produce natural milk which includes important proteins such as immunoregulatory proteins is important, and the feeding of the infant with the mother's milk can represent improvements in the care and health of the premature child.

The peptides of the present invention also have utility in the cessation of lactation in mothers where this is desired. Cessation of lactation in the mother may be desired, for example, following a still birth, where bottle feeding is the preferred method of feeding the newborn child, in elective weaning situations, or where the mother experiences discomfort caused by over production of milk and/or underfeeding by the child.

### Example 9

Utility in Dairy Milk Production

The artificial control of the lactation cycle is important both for enhancing milk production and for improving animal welfare.

The peptides of the present invention, in combination with antibodies directed to these peptides can be used to influence lactation by commercially bred and retained animals.

Optimisation of the milking regimen can be obtained through a combination of administering the peptides and antibodies thereto of the invention.

Specifically, antibodies can be administered to increase lactation rates during the milking period.

Conversely, commercially produced animals, such as milking cows are specifically bred to promote their high level milk producing ability. However, the turning off of milk production can be a problem.

The normally used method of drying-off dairy cows, goats and sheep is by the abrupt cessation of milking towards the end of the milk production cycle. The undesirable results of this technique are an increased susceptibility of the mammary gland tissues to infections and a swelling of the udder, which in turn causes the animals discomfort and pain.

Accordingly, administration of the peptides according to the present invention can assist in the cessation of milk secretion and improvements in the ‘drying-off’ period.

Further, the co-administration of the peptides and antibodies thereto would represent ethically acceptable husbandry practices.

Administration of the peptides of the present invention can also be used to induce stoppage of milk production as a preventative measure, in situations where a high risk of infection with pathogenic micro-organisms exists. Since administration can be specific, it is possible to direct administration to only the infected or ‘at risk’ teats in order to cause cessation of lactation there. Targeted administration in this way can allow uninterrupted milk production (fit for human consumption) from untreated quarters.

Such a treatment would be of particular use in the treatment of chronic repeated mastitis in ruminants caused by the bacteria Staphylococcus agalactiae or Staphylococcus aureus. Intra-mammary treatment of this condition with antibiotics is not an ideal solution. Other than the problems they cause with the milk (withdrawal for several days, contamination from antibiotic residues, problems associated with yoghurt and cheese processing), antibiotics are not always effective and in due course the organism may develop resistance to the penicillin and beta-lactam drugs currently preferred to treat this condition.

All documents referred to in this specification are herein incorporated by reference. Various modifications and variations to the described embodiments of the inventions will be apparent to those skilled in the art without departing from the scope of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A peptide including the amino acid sequence RPKHPIKHQG (SEQ ID NO: 1), AVAVSQEAN (SEQ ID NO: 2) or SEGVALDPAR (SEQ ID NO: 3) or an analogue thereof.

2. A peptide as claimed in claim 1 wherein SEQ ID NO:1, 2 or 3 is the N-terminal sequence of the peptide.

3. A peptide as claimed in claim 1 wherein the peptide can be co-purified with each of the other peptides including the amino acid sequence shown in SEQ ID NO: 1, SEQ ID NO: 2 or SEQ ID NO: 3 from a 6-30 kDa fraction of whey protein of cow's milk.

4. A peptide as claimed in claim 1 wherein the peptide, in combination with one or other of the peptides including the amino acid sequence of SEQ ID NO: 1, 2 or 3 reduces the level of milk production in a lactating cell.

5. A composition for influencing the rate of milk production by a lactating cell, the composition including a peptide including the sequence RPKHPIKHQG (SEQ ID NO:1) or an analogue thereof.

6. A composition for influencing the rate of milk production by a lactating cell, the composition including a peptide including the sequence AVAVSQEAN (SEQ ID NO: 2) or an analogue thereof.

7. A composition for influencing the rate of milk production by a lactating cell, the composition including a peptide including the sequence SEGVALDPAR (SEQ ID NO: 3) or an analogue thereof.

8. A composition for influencing the rate of milk production by a lactating cell, the composition including peptides including at least two of the sequences RPKHPIKHQG (SEQ ID NO: 1), AVAVSQEAN (SEQ ID NO: 2) or SEGVALDPAR (SEQ ID NO: 3) or analogues thereof.

9. A method of influencing milk secretion in animals, the method including the steps of administering at least one peptide including the amino acid sequence RPKHPIKHQG (SEQ ID NO: 1), AVAVSQEAN (SEQ ID NO: 2) or SEGVALDPAR (SEQ ID NO: 3) or an analogue thereof.

10. A method as claimed in claim 9 wherein the animal is selected from the group comprising a cow, goat and sheep.

11. A method as claimed in claim 9 wherein the animal is a human.

12. A method of modulating the milk secretion rate of a lactating cell, the method including the steps of:
selecting a composition including at least one peptide including the amino acid sequence RPKHPIKHQG (SEQ ID NO: 1), AVAVSQEAN (SEQ ID NO: 2) or SEGVALDPAR (SEQ ID NO: 3) or an analogue thereof,
administering the composition to the animal through the appropriate targeting of the mammary gland, and
exposing the cells of the mammary gland to a concentration of the composition sufficient to induce an inhibitory feedback mechanism which reduces lactation by the mammary gland cells.

13. A method as claimed in claim 12 wherein the composition is administered through intra-ductal administration.

14. A method as claimed in claim 12 wherein the delivery of the composition is by means of a bolus of peptide which is preferably encapsulated.

15. A method as claimed in claim 14 wherein the encapsulation material is an oil such as mineral oil.

16. A method as claimed in claim 12 wherein the composition is administered at a dose yielding a final concentration of peptides in milk in the range 0.01-1.6 micromolar.

17. (canceled)

18. A method of treating mastitis, the method including the step of administering a therapeutically useful amount of a at least one peptide including the amino acid sequence RPKHPIKHQG (SEQ ID NO: 1), AVAVSQEAN (SEQ ID NO: 2) or SEGVALDPAR (SEQ ID NO: 3) or an analogue thereof.

19. A composition for influencing lactation in animals, the composition including a peptide including the amino acid sequence RPKHPIKHQG (SEQ ID NO: 1), AVAVSQEAN (SEQ ID NO: 2) or SEGVALDPAR (SEQ ID NO: 3) or an analogue thereof.

20. A composition as claimed in claim 19 wherein the animal is a non-human animal.

21. A composition as claimed in claim 19 wherein the animal is a cow, goat or sheep.

22. An antibody which specifically binds a peptide including the amino acid sequence shown in SEQ ID NO: 1, SEQ ID NO: 2 or SEQ ID NO: 3 or analogues thereof.

23. An antibody which specifically binds a peptide consisting of the amino acid sequence shown in SEQ ID NO:1, SEQ ID NO: 2 or SEQ ID NO: 3 or analogues thereof.

24. (canceled)
